(19) United States
(12) Patent Application Publication
(10) Pub. No.: US 2007/0281291 A1
(43) Pub. Date: Dec. 6, 2007

(54) Method and Medium for the Rapid Detection of E.Coli in Liquid Samples

(76) Inventor: John Michael Kuchta, Pittsburgh, PA (US)

Correspondence Address: THORP REED & ARMSTRONG, LLP, ONE OXFORD CENTRE, 301 GRANT STREET, 14TH FLOOR, PITTSBURGH, PA, 15219-1425, US

(21) Appl. No.: 11/752,220

(22) Filed: May 22, 2007

Related U.S. Application Data
(60) Provisional application No. 60/802,549, filed on May 22, 2006.

(51) Int. Cl. C12Q 1/00

(52) U.S. Cl. 435/4

(57) ABSTRACT

A novel method and media for the rapid detection of E. coli bacteria in liquid samples is disclosed. This new replica-plating method allows for preservation of the initial sample and the elimination of inhibiting factors. The new induction media permits rapid detection of E. coli due to the fact that it is non-nutritional and is primarily being used to increase induction of the genes associated with overall catabolism of the carbohydrate and not growth per se. The end result is quicker results.

### CROSS REFERENCE TO RELATED APPLICATIONS

This application claims priority to U.S. Provisional Application Ser. No. 60/802,549, filed on May 22, 2006, which is hereby incorporated into this disclosure in its entirety.

### BACKGROUND—FIELD OF THE INVENTION

Fecal coliform bacteria, such as E. coli, are found naturally in the intestines of humans and animals. If ingested, however, a toxin that his bacteria produces can cause damage to red blood cells, kidney and other organs and can cause acute kidney failure or even death in individuals with compromised or weak immune systems.

Of all types of coliform bacteria, studies have shown that E. coli presence is the most reliable indicator of fecal contamination in potable and recreational waters. Its presence is also closely monitored in various other industries that suspect fecal pollution, including dairy farming and produce and vegetable preparation and distribution

In order to detect E. coli and other fecal coliforms, it is well known in the industry to add 4-methylumbelliferyl-•-D-glucuronide (MUG) to a growth medium to detect the bacterial enzyme •-glucuronidase (GUS) because GUS interacts with MUG to create a byproduct, 4-methylumbelliferone (4-MU), that emits a fluorescence and is thus easy to confirm because none of the constituents to the reaction are fluorescent by themselves. The importance of detecting GUS is that most strains of E. coli, in addition to some strains of Salmonella and Shigella, produce the GUS enzyme. Since both Salmonella and Shigella are also dangerous if ingested, their detection provides additional information in determining the potability of drinking water, for example.

Using existing approved methods, samples taken from a suspect water source are vacuum-forced through a sterile membrane filter (HAWG 047, Millipore Corp) via a sterile stainless steel funnel in order to collect any coliform bacteria in the water. The filter is then placed in a petri dish containing mEndo LES agar and incubated for 24 hours at 35° C. After this initial incubation period, a sterile swab is used to collect bacteria from all colonies that have grown on the agar. This swab is then dipped in test tubes containing one of two media: Lauryl Tryptose or Brilliant Green Bile Broth. After an additional 18 to 24 hour period, the test tubes are examined to determine if they are turbid. If both the Lauryl Tryptose and the brilliant green bile broth are turbid, and additional test is performed to confirm the presence of E. coli. In this final test, a swab is run over all of the colonies on the original filter and dipped into a test tube containing EC, a medium developed especially for the detection of E. coli, and MUG. If, at any point in the next twenty-four (24) hours at 44° C., the liquid in this final test tube becomes fluorescent under long wavelength (366 nm) UV light, the testing is positive for E. coli. In cases with a significant population of E. coli, fluorescence can be detected in as little as 7 to 8 hours, however, this existing approved procedure still takes three to four days to produce results because it depends on the growth of additional colonies.

Alternatively, it is known to perform the above tests simultaneously, but this practice suffers from the fact that it adds unnecessary expense in cases where E. coli is not present because the third stage in the existing process will not be performed if the results of the second stage are negative. Moreover, performing the existing procedures simultaneously still requires a forty-eight hour time period.

It is also well-known to prepare m-Endo LES agar and incubate sample filters for twenty to twenty-four hours at 35° C. to allow colonies to form. Then, the original filter is transferred to a nutrient agar that also contains MUG. This original filter in the new agar is then incubated for an additional four hours to determine whether any GUS is present. A disadvantage of this method, however, is that the fluorescent halos develop slowly in the nutrient agar and can be difficult to detect. Inhibition of fluorescence has also been observed around some MUG positive colonies on m-Endo LES agar plates. This undesirable result is possibly caused by a component of the medium that inhibits fluorescence production.

It is desirable, therefore, to have a process and medium which provides accurate results for the presence or absence of E. coli in a liquid sample in a shorter amount of time than is currently possible. It would be preferable if the new process would be amenable to being performed with very little transference of original growth media, thus lowering the risk of contamination by inhibitors for the reaction. It would also be preferable if the new process were effective in transferring colonies regardless of the growth medium employed.

### SUMMARY OF THE INVENTION

The present invention overcomes the disadvantages of prior art methods by introducing the use of a replica-plating technique that, when preformed in conjunction with a simple non-nutritional buffered medium containing MUG, verifies the presence of E. coli in a water sample in as little as 30 minutes after performing the technique, all without damaging the sample filter. E. coli colonies easily pass from the sample filter to the transfer filter during the procedure of the present invention. When the transfer filter containing the transferred bacterial colonies is then placed upon a MUG-containing agar, the GUS enzyme created by the E. coli bacteria hydrolyzes the MUG to produce the fluorescent by-product that is easily detectable.

The induction media is easy to make and can be stored for up to a year at 4° C. without significant degradation or decrease in reliability.

It is an advantage of the present invention that any growth medium supporting E. coli can be used as a source for this bacteria. Thus, a non-specific plate, such as m-HPC, can easily be checked to detect the presence of E. coli.

In another form of the above-identified inventive medium, a second inducer is applied to the MUG-containing agar in order to maximize production of the GUS enzyme, which, in turn, speeds the confirmation of the presence of E. coli bacteria. Alternative non-physiological substrates (competitive inhibitors) have a different affinity for the active site of GUS. This is a common phenomenon especially among regulatory enzymes. Therefore, addition of a low concentration of a superior inducer enhances the speed as well as the intensity of the reaction. The second inducer, 4-nitrophenyl-beta-D-alucuronide, was found to be very effective for induction of the GUS enzyme.

The above and other objects, features and advantages of the instant invention will be apparent in the following detailed description of the preferred embodiment thereof when read in conjunction with the accompanying drawings.

### BRIEF DESCRIPTION OF THE DRAWINGS

FIG. 1 is a photograph of two plates, the plate on the left having colonies of E. coli grown on a growth medium after being incubated at 44.5° C. for 24 hours in accordance with known methods. The plate on the right is an unused induction medium prepared in accordance with a preferred method of the present invention.

FIG. 2 is a photograph of a transfer filter being placed on the growth medium containing the E. coli colonies.

FIG. 3 is a photograph that demonstrates the tamping down of the transfer filter to ensure colony transfer.

FIG. 4 is a photograph that demonstrates the proper method of removing the transfer filter from the growth medium after tamping down.

FIG. 5 is a photograph that demonstrates the placement of the transfer filter in the induction medium.

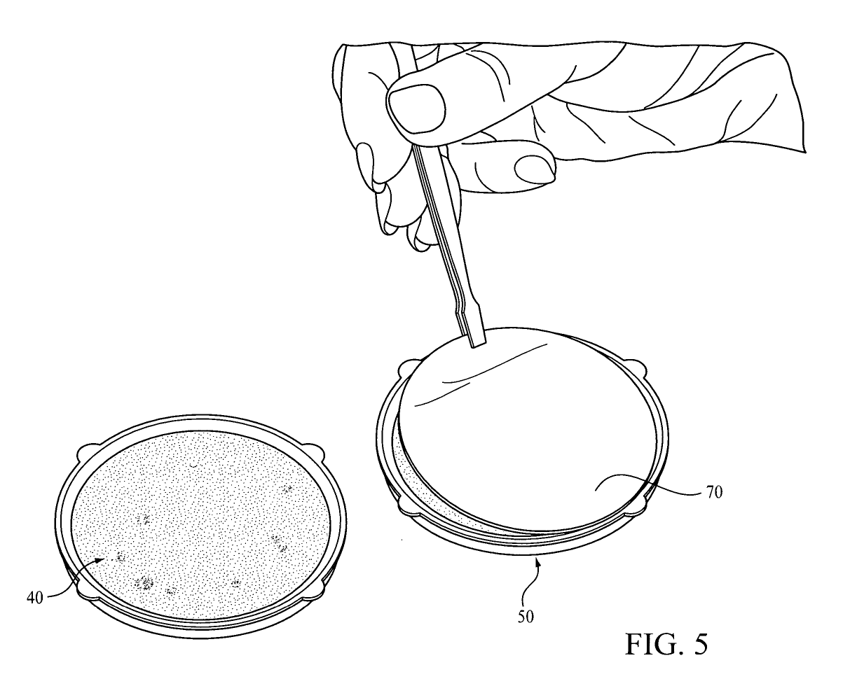

FIG. 6 is a photograph that demonstrates the transfer filter in place on the induction medium.

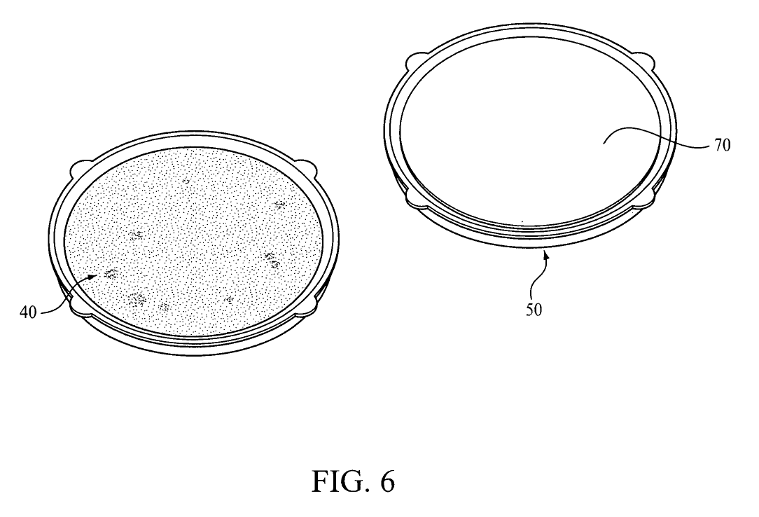

FIG. 7 is a photograph demonstrating the removal of the transfer filter from the induction medium.

FIG. 8 is a photograph that demonstrates the induction medium under long wavelength light after 45 minutes.

FIG. 9 is a photograph that demonstrates the induction medium under long wavelength light after 90 minutes.

### DESCRIPTION OF THE PREFERRED EMBODIMENT

Throughout the specification, the term “comprising” is used inclusively, in the sense that there may be other features and/or steps included in the invention not expressly defined or comprehended in the features or the steps specifically defined or described. What such other features and/or steps may include will be apparent from the specification read as a whole.

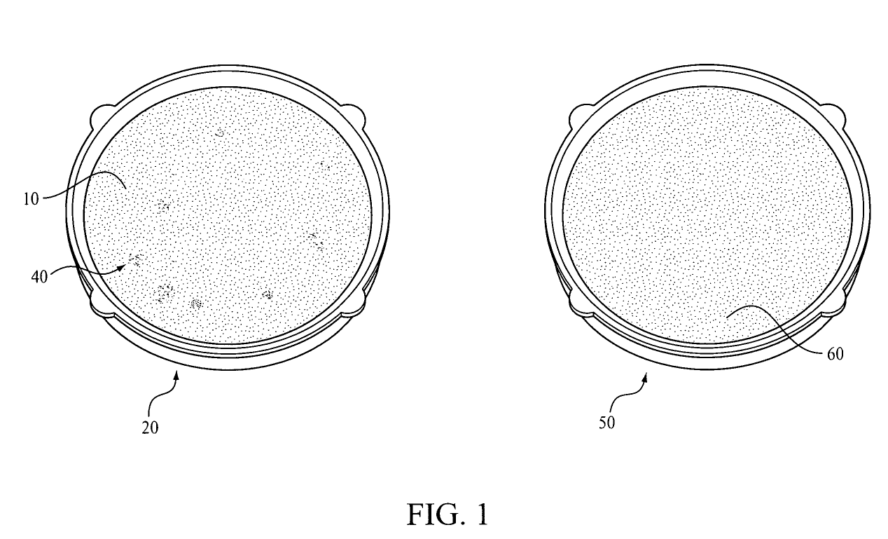

Referring now to FIG. 1, in accordance with standard, well-known procedures, water to be tested is vacuum forced through a sample filter 10, preferably a 47 mm filter HAWG, (Millipore, Bedford, Mass.) with a pore size of 0.45•m. This sample filter 10 is then placed on a growth place 20 and incubated for 18 to 24 hours at 35° C., using a growth medium (not shown) such as m-Endo LES according to well-known procedures. While other growth media such as mFC and mHPC could also be used, m-Endo LES had the best combination of speed and cost.

FIG. 1 demonstrates a growth plate 20 subsequent to incubation with bacterial colonies 40 visible on the sample filter 10. Also shown in FIG. 1 is an induction plate 50 containing an induction media 60.

In one preferred embodiment of the induction media 60 of the present invention, it comprises, per 100 ml: 0.05 M potassium phosphate buffer pH 7.2; 1.4 g agar, 0.5 g MUG and 0.25 g 4-nitrophenyl beta-D-glucuronide (4-NBG). To create the induction plate 50, the buffer and agar are added to 100 ml of filtered water, heated to dissolve constituents, and autoclaved for 5 minutes. MUG and 4-NBG are added as solids as soon as the autoclaving procedure is finished. After stirring, approximately 6 ml of this induction media 60 is dispensed into the induction plate 50, which is a tight fitting 50 mm plate.

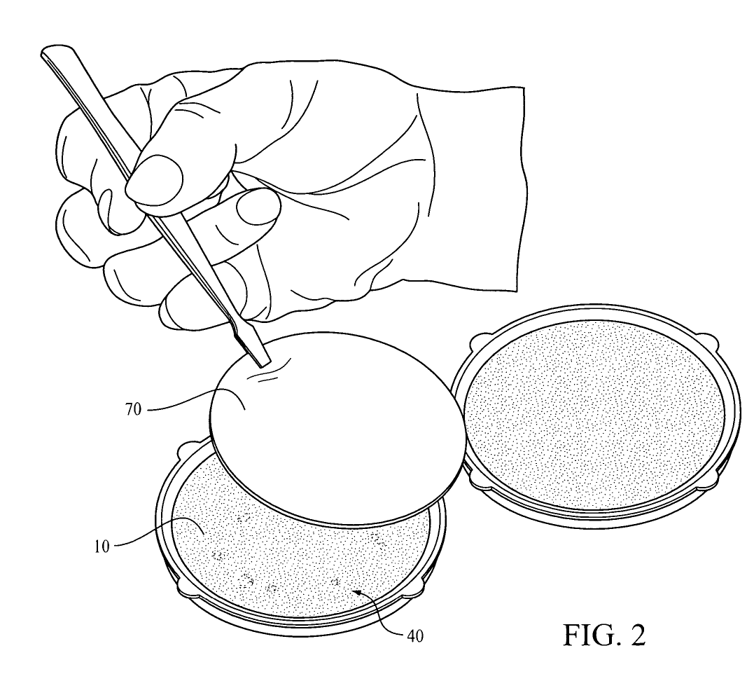

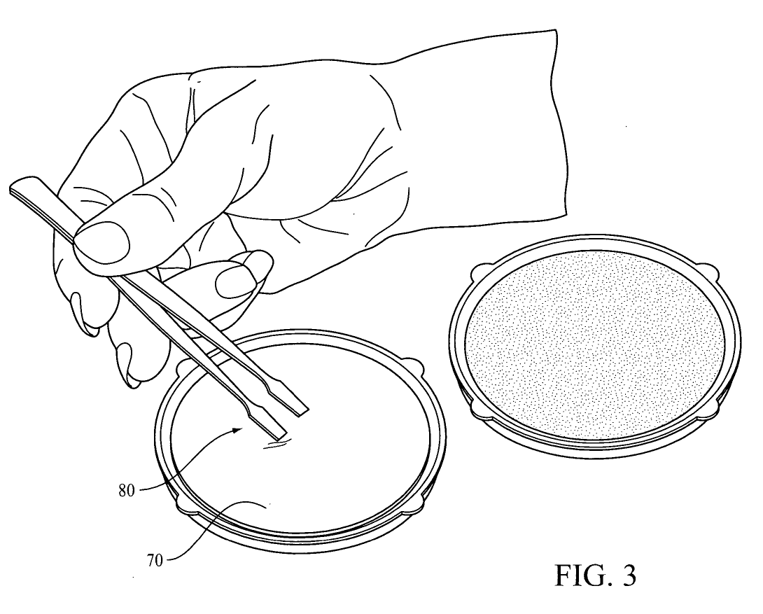

After incubation, FIGS. 2 and 3 demonstrate a transfer filter 70 being carefully placed grid side down on top of the sample filter 10 and being gently tamped down as shown in FIG. 3 onto the sample filter 10 and growth medium with flat-edged forceps 80 to assure good contact between the transfer filter 70 and the bacterial colonies 40 that have grown on the sample filter 10.

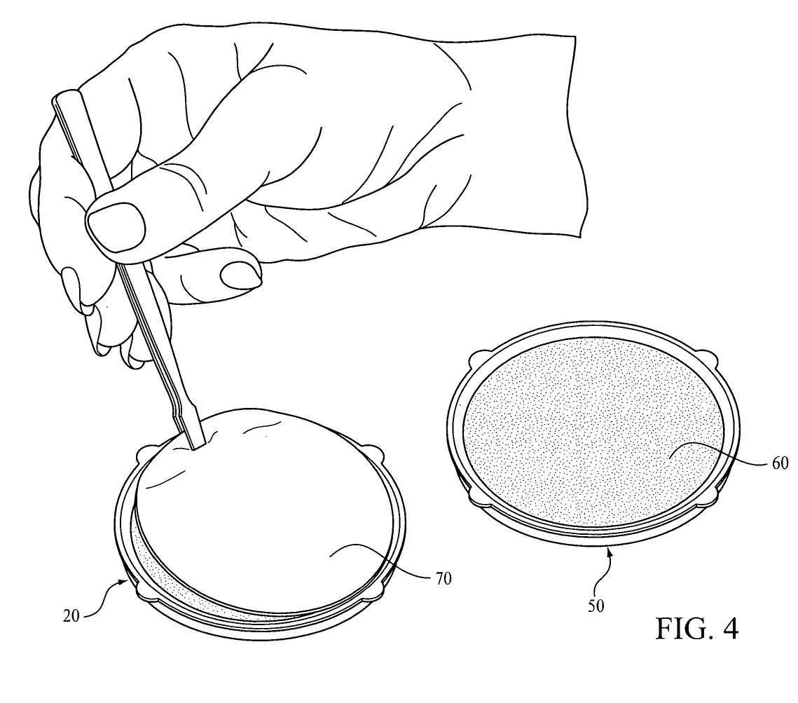

Next, as demonstrated in FIGS. 4,5 and 6, the transfer filter 70 is removed from the growth plate 20 and placed on the induction plate 50 containing and induction media 60 of the present invention and tamped down on the induction media to assure complete transfer of the bacterial colonies 40.

When tamping the transfer filter 70 down onto the induction media 60, sufficient contact must be ensured to facilitate transfer. This can either be done by leaving the transfer filter 70 on the induction media 60 for a time period preferably in the range of thirty (30) seconds to five (5) minutes or by ensuring that the entire transfer filter 70 has become visibly moist. Once contact has been verified, the transfer filter 70 can then either be saved for later additional testing or discarded.

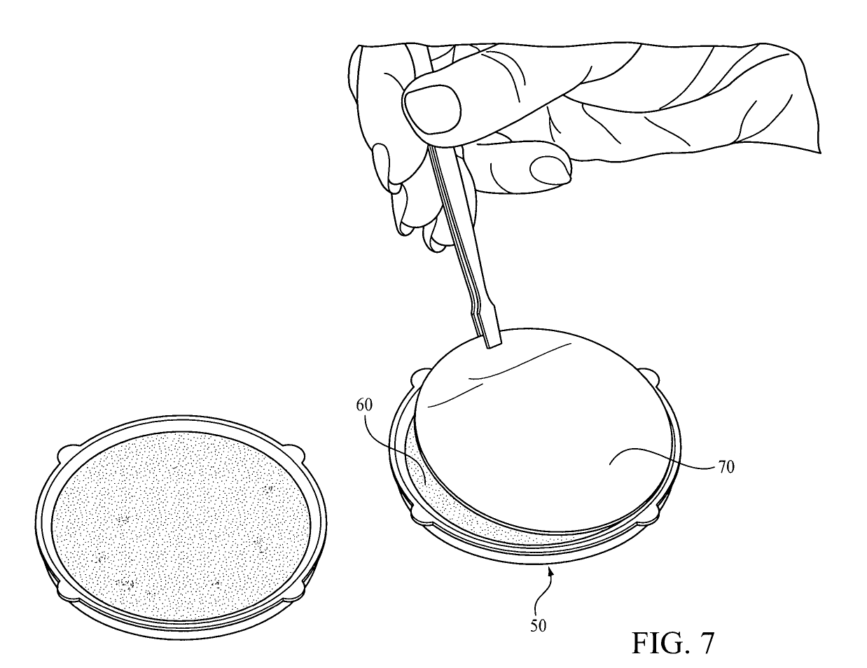

FIG. 7 demonstrates the removal of the transfer filter 70 from the induction plate 50. The induction media 60 is then incubated at 35° C. for 0.5 to 3.5 hrs.

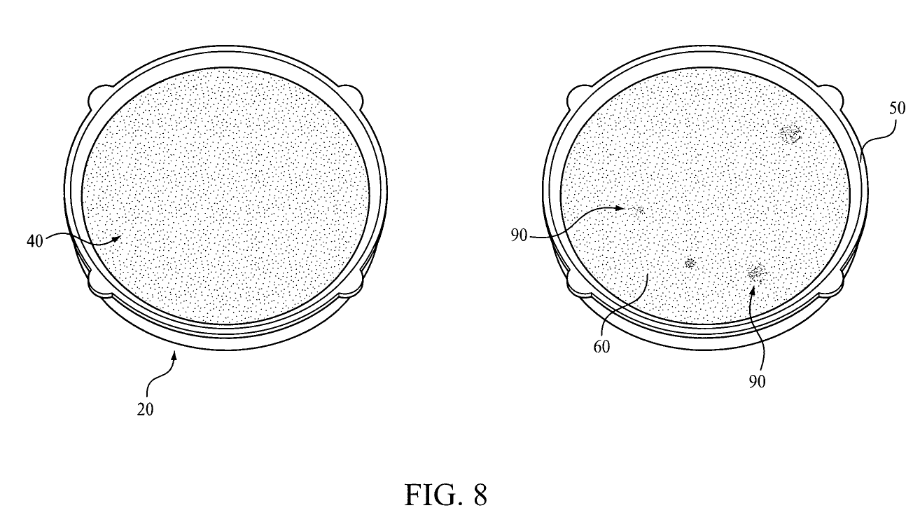

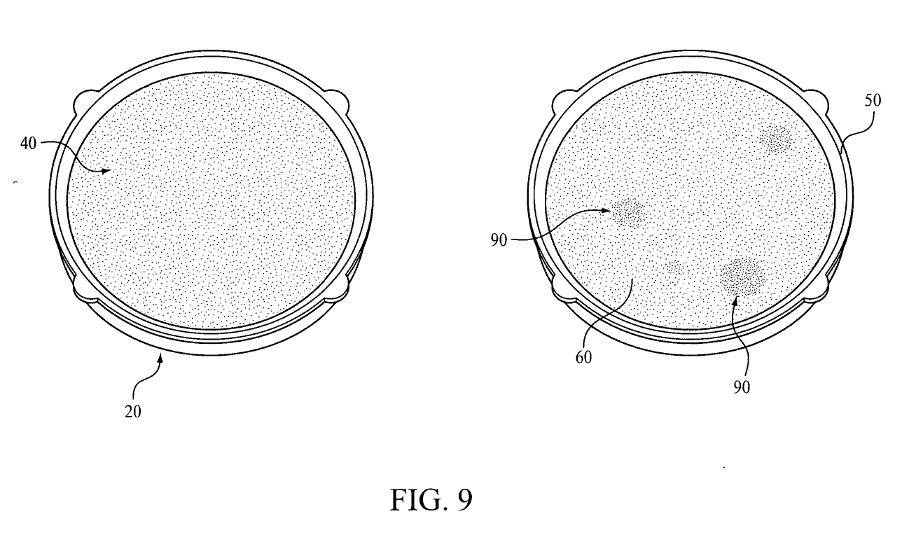

FIG. 8 shows the induction plate 50 under long wavelength UV (366 nm) light after forty-five (45) minutes of incubation and FIG. 9 shows the same induction plate 50 after ninety (90) minutes of incubation. The fluorescent spots 90 were created by transferred E. coli colonies cleaving the MUG and creating 7-hydroxy-4-methylcoumarin (MU), the fluorescent byproduct that is visible when exposed to long-wavelength ultraviolet light. If a qualitative analysis is required, the fluorescent spots 90 on the induction media 60 can be correlated to bacterial colonies 40 on the growth plate 20 and confirmed as E. coli.

Since this new method and medium does not require separate bacterial colony growth, it offers screening for E. coli in less time and at a lower cost to the laboratory than existing methods and media. Further, this procedure is highly selective for E. coli since it relies on the generation of an enzyme produced chiefly by that bacteria. MUG cannot be placed in the growth media itself because of rapid diffusion of fluorescence.

When known samples of E. coli were tested using the method and media of the present invention, fluorescence was exhibited quickest with mFC plates (usually within 30 minutes) followed by mEndo LES and NB plates, which took up to 3.5 hrs for development. The method and media of the present invention did, however, prove to be extremely reliable and accurate as the results set forth in Table 1 demonstrate:

TABLE 1
% Recovery of E. coli from m-ENDO LES Growth Plates

| | | mEndo Plate | (duplicate) | Induction plate | (duplicate) | |
|---|---|---|---|---|---|---|
| | % Recovery | # positive | # positive | # positive | # positive | Description |
| 1 | 0/0 | 110 | 161 | 0 | 0 | 0157:h7 E. coli |
| 2 | 0/0 | 102 | ND | 0 | ND | 0157:H7 E. coli |
| 3 | 0/0 | 58 | | 0 | 0 | Salmonella |
| 4 | 100/98 | 77 | 43 | 77 | 42 | E. coli |
| 5 | 99/100 | 86 | 52 | 85 | 52 | E. coli |
| 6 | 100/99 | 122 | 99 | 122 | 98 | E. coli |
| 7 | 98/98 | 1108 | 55 | 106 | 54 | E. coli |
| 8 | 0/0 | 32 | 66 | 0 | 0 | E. coli |
| 9 | 0/0 | 87 | 44 | 0 | 0 | E. coli |
| 10 | 94/97 | 34 | 57 | 32 | 55 | E. coli |
| 11 | 100/99 | 76 | 84 | 76 | 83 | E. coli |
| 12 | 98/100 | 62 | 52 | 61 | 52 | E. coli |
| 13 | 96/97 | 26 | 37 | 25 | 36 | E. coli |
| 14 | 97/98 | 66 | 53 | 64 | 52 | ATCC 35218 E. coli |
| 15 | 97/99 | 115 | 88 | 112 | 87 | ATCC 1029 E. coli |
| 16 | 0/0 | 136 | 118 | 0 | 0 | ATCC 8739 E. coli |
| 17 | 0/0 | 90 | 75 | 0 | 0 | ATCC 35150 E. coli |
| | initial/duplicate | | | | | |

As seen in Table 1, known E. coli strains were tested using the method and medium of the present invention. When the numbers of sheen or dark red colonies on mEndo LES media was compared to those fluorescing on the induction plates of the present invention, it can be seen that nearly 100% recovery was achieved when the bacteria is MUG positive. In the case of some enterohemorrhagic strains and the Salmonella strain referenced in the table, the present invention does not work with MUG negative bacteria and other tests are known for those strains.

It was also discovered, during the course of evaluating the present invention, that the detection of halos and spots surrounding mEndo sheen colonies when the whole filter was transferred to a NB medium containing MUG significantly reduces the carryover of inhibitory components of mEndo LES medium and adds clarity to the plates containing MUG. This resulted in the appearance of sharp, distinct fluorescent spots.

Prior art references have suggested not using lactose-based media in conjunction with MUG since acidification may reduce fluorescence. However, the new method and medium of the present invention have mitigated this concern since the new media is primarily being used to increase induction of the genes associated with overall catabolism of this carbohydrate and not growth per se.

Since many modifications, variations and changes in detail can be made to the described preferred embodiment of the invention, it is intended that all matters in the foregoing description and shown on the accompanying drawings be interpreted as illustrative and not in a limiting sense. It will be readily apparent to those skilled in the art that the method and media of the instant invention can easily be modified to be used with other experimental protocols as well. The scope of the invention should be determined by the claims and their legal equivalents.

I claim:

1. A method of detecting E. coli bacteria in a sample material comprising
(a) placing a second filter in contact with a first filter previously treated with the sample material and incubated;
(b) removing the second filter from contact with the first filter;
(c) placing a treated surface of the second filter into contact with an induction medium for a transferral time;
(d) removing the treated surface of the second filter from contact with the induction medium;
(e) incubating the induction medium for an incubation time; and
(f) exposing the induction medium to long wavelength light after the incubation time; and
(g) observing fluorescence indicating the presence of E. coli.

2. The method of claim 1, wherein the induction media includes a substance that yields a detectable byproduct in the presence of an enzyme produced by E. coli.

3. The method of claim 2, wherein the detectable byproduct emits fluorescence.

4. The method of claim 2, wherein the detectable byproduct comprises 7-hydroxy-4-methylcoumarin.

5. The method of claim 1, wherein both the first filter and the second filter are comprised of membrane filters.

6. The method of claim 1, wherein the induction media includes 4-methylumbelliferyl-•-D-glucuronide.

7. The method of claim 1, wherein the induction media includes a substance that facilitates the production of •-glucuronidase by E. coli.

8. The method of claim 1, wherein the induction media comprises, per 100 ml: 0.05 M potassium phosphate buffer pH 7.2, agar, 0.5 gm 4-methylumbelliferyl-•-D-glucuronide, 0.25 gm 4-methyl-beta-D-glucuronide and water.

9. The method of claim 1, wherein the transferral time is an amount of time in the range of from about thirty seconds to about five minutes.

10. The method of claim 1, wherein the incubation time is an amount of time in the range of from about thirty minutes to about three hours.

11. An induction medium for use in detecting E. coli comprising a substance that yields a detectable byproduct in the presence of an enzyme produced by E. coli.

12. The induction medium of claim 11, wherein the detectable byproduct emits long wave ultraviolet fluorescence.

13. The induction medium of claim 11, wherein the detectable byproduct comprises 7-hydroxy-4-methylcoumarin.

14. The induction medium of claim 11, further comprising a substance that facilitates the production of •-glucuronidase by E. coli.

15. The induction medium of claim 14, wherein the substance is cyanide.

16. The induction medium of claim 11 wherein the substance is comprised of, per 100 ml: 0.05 M potassium phosphate buffer pH 7.2, agar, 0.5 gm 4-methylumbelliferyl-•-D-glucuronide, lactose, 4-nitrophenyl-beta-D-glucuronideand water.

17. A method of detecting E. coli bacteria in a liquid sample comprising:
(a) passing the liquid sample through a first filter;
(b) placing the first filter in contact with a nutrient medium;
(c) incubating the first filter for an incubation time;
(d) placing a second filter in contact with the first filter;
(e) placing a treated surface of the second filter in contact with an induction medium for a transferral time;
(f) removing the treated surface of the second filter from the induction medium;
(g) incubating the induction medium for a second incubation time;
(h) applying a long wave ultraviolet light to the induction medium after the second incubation time; and
(i) observing fluorescence indicating the presence of E. coli bacteria.

18. The method of claim 17, wherein the second incubation time is an amount of time in the range of from about thirty minutes to about three hours.

19. An induction medium for detecting E. coli comprising, per 100 ml: 0.05 M potassium phosphate buffer pH 7.2, agar, 0.5 gm 4-methylumbelliferyl-•-D-glucuronide, 0.25 gm 4-methyl-beta-D-glucuronide and water.

20. A method of detecting E. coli bacteria in a sample material comprising
(a) placing a second filter in contact with a first filter previously treated with the sample material and incubated;
(b) removing the second filter from contact with the first filter;
(c) placing a treated surface of the second filter into contact with an induction medium for a transferral time, said induction medium comprising, per 100 ml: 0.05 M potassium phosphate buffer pH 7.2, agar, 0.5 gm 4-methylumbelliferyl-•-D-glucuronide, 0.25 gm 4-methyl-beta-D-glucuronide and water;
(d) removing the treated surface of the second filter from contact with the induction medium;
(e) incubating the induction medium for an incubation time; and
(f) exposing the induction medium to long wavelength light after the incubation time; and
(g) observing fluorescence indicating the presence of E. coli.
